(19) United States
(12) Patent Application Publication
(10) Pub. No.: US 2014/0213995 A1
(43) Pub. Date: Jul. 31, 2014

(54) Urostomy Drainage Appliance

(71) Applicant: John Roy GARRETTSON, Kerrville, TX (US)

(72) Inventor: John Roy GARRETTSON, Kerrville, TX (US)

(21) Appl. No.: 14/165,776

(22) Filed: Jan. 28, 2014

Related U.S. Application Data
(60) Provisional application No. 61/758,895, filed on Jan. 31, 2013.

(51) Int. Cl. A61F 5/448

(52) U.S. Cl. CPC A61F 5/448
USPC 604/344

(57) ABSTRACT

A urostomy drainage appliance is an apparatus that improves upon existing urostomy bag systems. The urostomy drainage appliance accomplishes this through the user of a convex drainage appliance that directs the flow of excreted urine through a set of a drainage stems. The drainage stems divert urine from the convex drainage appliance through an attached drainage hose into a remotely positioned collection bag. The urostomy drainage appliance utilizes particularly positioned drainage stems to allow a user to sleep on their side without restricting urine from flowing into the collection bag. The convex drainage appliance is particularly formed permitting facilitated concealment while worn in public. The urostomy drainage appliance can be flushed using the drainage stems reducing bacterial growth and reducing the frequency the individual replaces the urostomy appliance.

The current application claims a priority to the U.S. Provisional Patent application Ser. No. 61/758, 895 filed on Jan. 31, 2013.

### FIELD OF THE INVENTION

The present invention relates generally to stomas due to the removal of a bladder. More specifically, the present invention is a cut-to-fit urostomy dome system which improves the flow of urine form a stoma due to the removal of the bladder.

### BACKGROUND OF THE INVENTION

As a consequence of a medical illness or trauma related injury, an individual may have their bladder replaced with an externally positioned prosthetic collection system. These ostomy collection systems are externally mounted receptacle that are attached to an abdominal stoma connected to the individual's urinary tract system. These ostomy collection systems allow for the drainage of urine to be directed through the stoma and into a urostomy bag. These existing urostomy bag systems are connected to an individual through the use of a wafer which is mounted to the peristomal region surrounding an individual's stoma. The wafer functions as a mounting point that seals the urostomy bag against the stoma.

While urostomy bag systems have been around for some time, they are plagued with several disadvantages that have yet to be adequately addressed. One disadvantage is that urostomy bags systems cannot be adequately cleaned out. Resultantly, urostomy bag systems require an individual to frequently replace their urostomy bag in order to avoid urinary tract infections. Another disadvantage is that urostomy bag system lack discretion when worn by an individual in public. As a result of their bulky construction and their direct attachment to the individual's stoma, urostomy bag system are difficult to cover up without multiple layers of clothing. Still another disadvantage associated with existing urostomy bag system is due to restricting an individual's sleeping position. As a result of attaching the urostomy bag directly to the wafer, the individual is restricted to sleeping on their back in order to prevent their stoma from being occluded or the urostomy bag being pinched restricting the flow of urine into the urostomy bag. Although some of the aforementioned disadvantages are inherently associated with an abdominal stoma, many aspects can be solved or improved upon through a redesign of the existing urostomy bag system.

It is therefore an object of the present invention to provide a urostomy drainage appliance that improves upon existing urostomy bag systems. The urostomy drainage appliance accomplishes this through the user of a convex drainage appliance that directs the flow of excreted urine through a set of a drainage stems. The drainage stems divert urine from the convex drainage appliance through an attached drainage hose into a remotely positioned collection bag. The urostomy drainage appliance utilizes particularly positioned drainage stems to allow a user to sleep on their side without restricting urine from flowing into the collection bag. The convex drainage appliance is particularly formed permitting facilitated concealment while worn in public. The urostomy drainage appliance can be flushed using the drainage stems reducing bacterial growth and reducing the frequency the individual replaces the urostomy appliance.

### BRIEF DESCRIPTIONS OF THE DRAWINGS

FIG. 1 is a frontal perspective view displaying the urostomy drainage appliance with an attached drainage hose configured in the current embodiment of the present invention.

FIG. 2 is an expanded view displaying the convex drainage appliance detached from the appliance mount and the wafer in the current embodiment of the present invention.

FIG. 3 is an expanded view displaying the convex drainage appliance, the appliance mount, the appliance mount coupler, and the wafer in the current embodiment of the present invention.

FIG. 4 is an expanded view displaying the lower sections of the convex drainage appliance, the appliance mount, the appliance mount coupler, and the wafer in the current embodiment of the present invention.

FIG. 5 is an enhanced sectional view displaying the internal component arrangement between the appliance mount, the convex drainage appliance, and the wafer in the current embodiment of the present invention.

FIG. 6 is an expanded sectional view displaying the internal component distributions between the appliance mount, the convex drainage appliance, the at least two drainage stems, and the wafer in the current embodiment of the present invention.

FIG. 7 is a top down elevational view displaying the angular positioning between the first drainage stem and the second drainage stem as per the current embodiment of the present invention.

FIG. 8 is an expanded view displaying the convex drainage appliance integrated with the appliance mount as per an embodiment of the present invention.

FIG. 9 is an expanded sectional view displaying the convex drainage appliance integrated with the appliance mount as per an embodiment of the present invention.

### DETAIL DESCRIPTIONS OF THE INVENTION

All illustrations of the drawings are for the purpose of describing selected versions of the present invention and are not intended to limit the scope of the present invention.

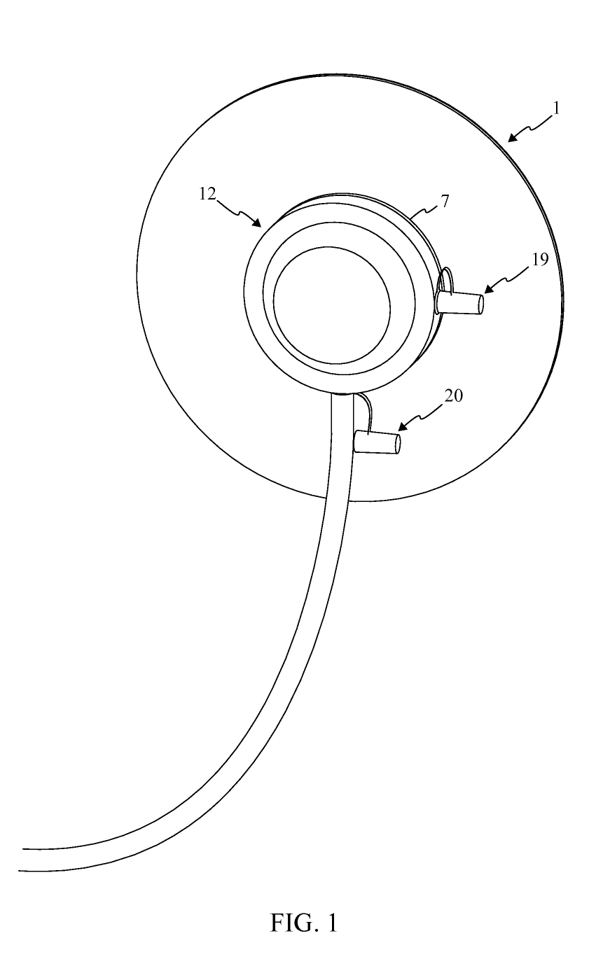

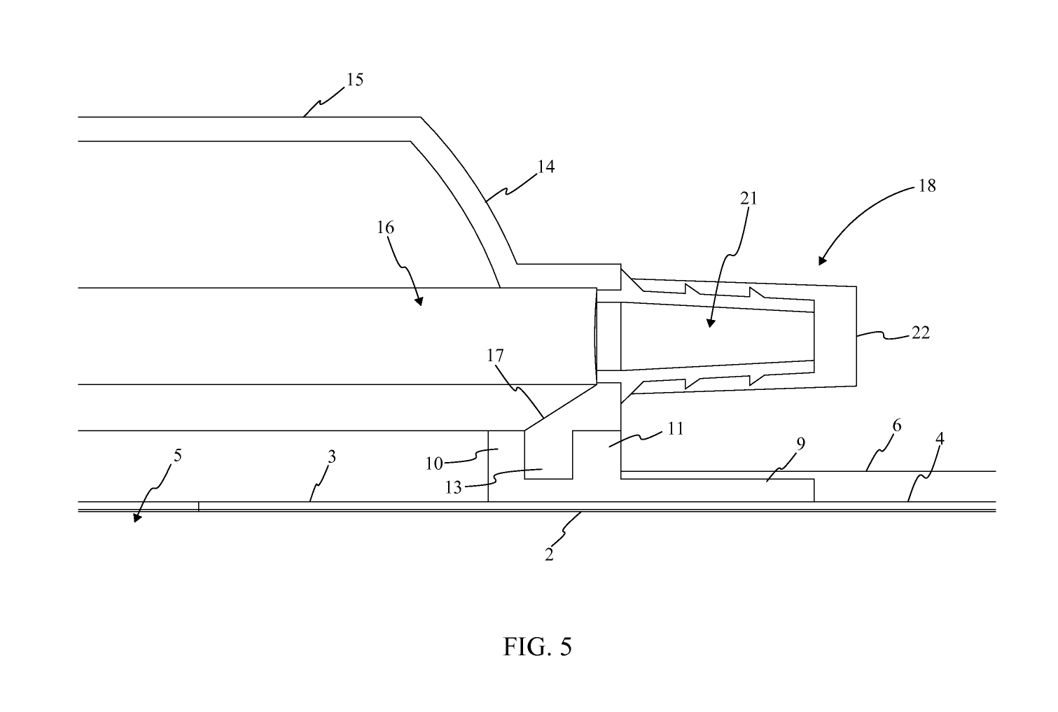

Referencing FIG. 1 and FIG. 5, the present invention is a urostomy drainage appliance that directs urine from a stoma into a drainage receptacle. The urostomy drainage appliance permits the drainage of the urine while a user is lying down on their side. In the current embodiment of the present invention, the urostomy drainage appliance comprises a wafer 1, an appliance mount 7, a convex drainage appliance 12, and at least two drainage stems 18. The wafer 1 is a barrier that adheres around the stoma and provides an attachment point for the appliance mount 7. The appliance mount 7 functions as a fixed base for the convex drainage appliance 12. The convex drainage appliance 12 forms an enclosure with the appliance mount 7 that directs the flow of urine from the stoma to the at least two drainage stems 18. The at least two drainage stems 18 are particularly positioned fluid outlets that drain urine from within the enclosure to a separate receptacle attached through a drainage hose.

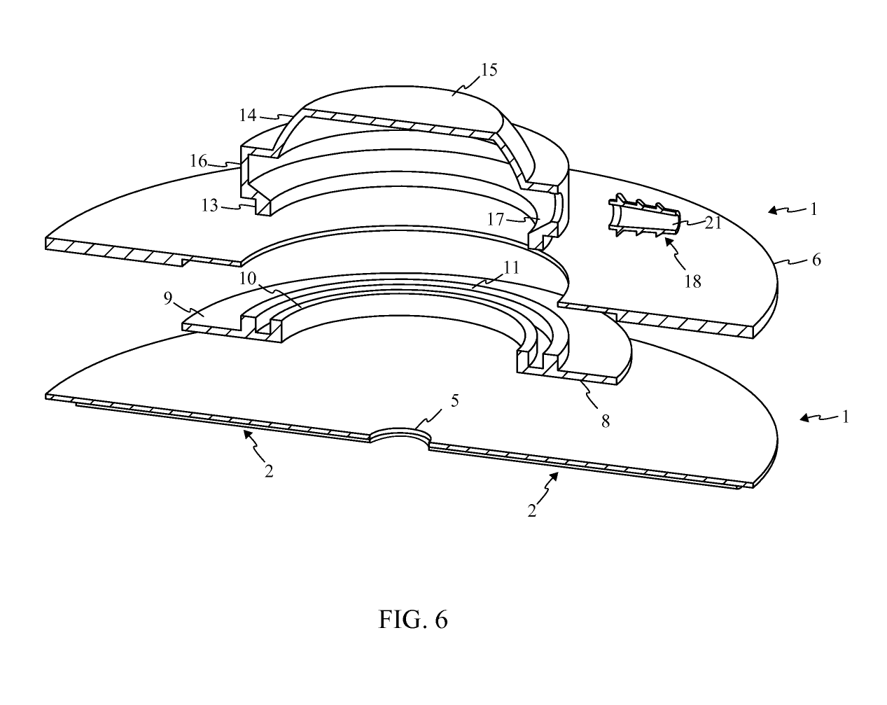

Referencing FIG. 5 and FIG. 6, the wafer 1 is a baseplate that enables the attachment of the urostomy drainage device to a user's body. The wafer 1 is perimetrically positioned on the peristomal region of a user's body. The wafer 1 is adhered to a user's skin forming a barrier that protects the prolonged exposure with excretions such as urine. In the current embodiment of the present invention, the wafer 1 comprises an adhesive interface 2, a stoma opening 5, an inner section 3, an outer section 4, and an appliance mount coupler 6. The adhesive interface 2 is the portion of the wafer 1 that is coupled to peristomal region of the user. The stoma opening 5 is an aperture in the wafer 1 that is coincident with a user's stoma. The inner section 3 and the outer section 4 are regions of the wafer 1 found opposite the adhesive interface 2. The inner section 3 is the region of the wafer 1 that is proximally positioned to the stoma opening 5. The inner section 3 and the outer section 4 are separated by the positioning of the appliance mount 7. The outer section 4 is the region that is found surrounding the appliance mount 7. The appliance mount coupler 6 is the engagement means that secures the appliance mount 7 to the wafer 1.

The inner section 3 of the wafer 1 in the current embodiment of the present invention is provided with a flexible construction. The flexible construction is provided as a means of accommodating and preventing ostomy related complications such as parastomal hernias from obstructing or restricting the drainage of excretion through the stoma opening 5. Parastomal hernias are abdominal protrusions that occur at the site of or immediately adjacent of the stoma. By providing the inner section 3 with a flexible construction, parastomal hernias are less likely to constrict around the stoma, restricting drainage of excretions through the urostomy drainage appliance. It should be noted that the flexible construction of the inner section 3 is provided with sufficient flexibility to prevent stomal strangulation in mild and minor cases while having sufficient rigidity to prevent a parastomal hernia from obstructing itself against the convex drainage appliance 12.

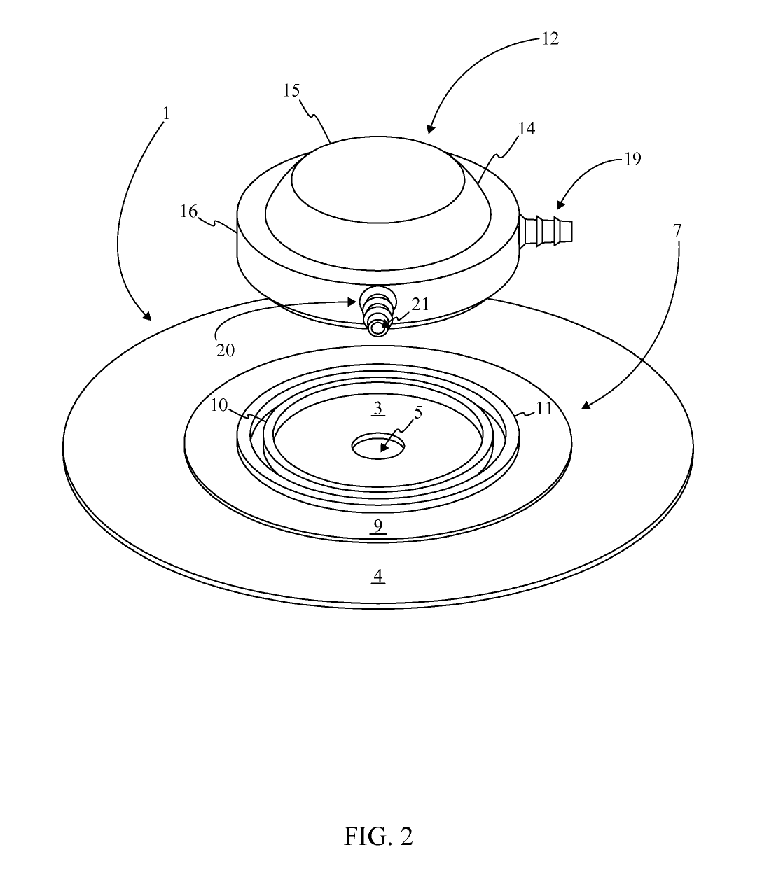

Referencing FIG. 2, the inner section 3 of the wafer 1 in the current embodiment of the present invention is customizable by a user, wherein the customizable nature of the inner section 3 allows a user to widen the stoma opening 5 by cutting away portions of the inner section 3 immediately adjacent to the stoma opening 5. It should be noted that due to the positioning of the adhesive interface 2 relative to the inner section 3 and the outer section 4, the user would cut into the a portion of the adhesive interface 2 when cutting into the inner section 3 immediately adjacent to the stoma opening 5.

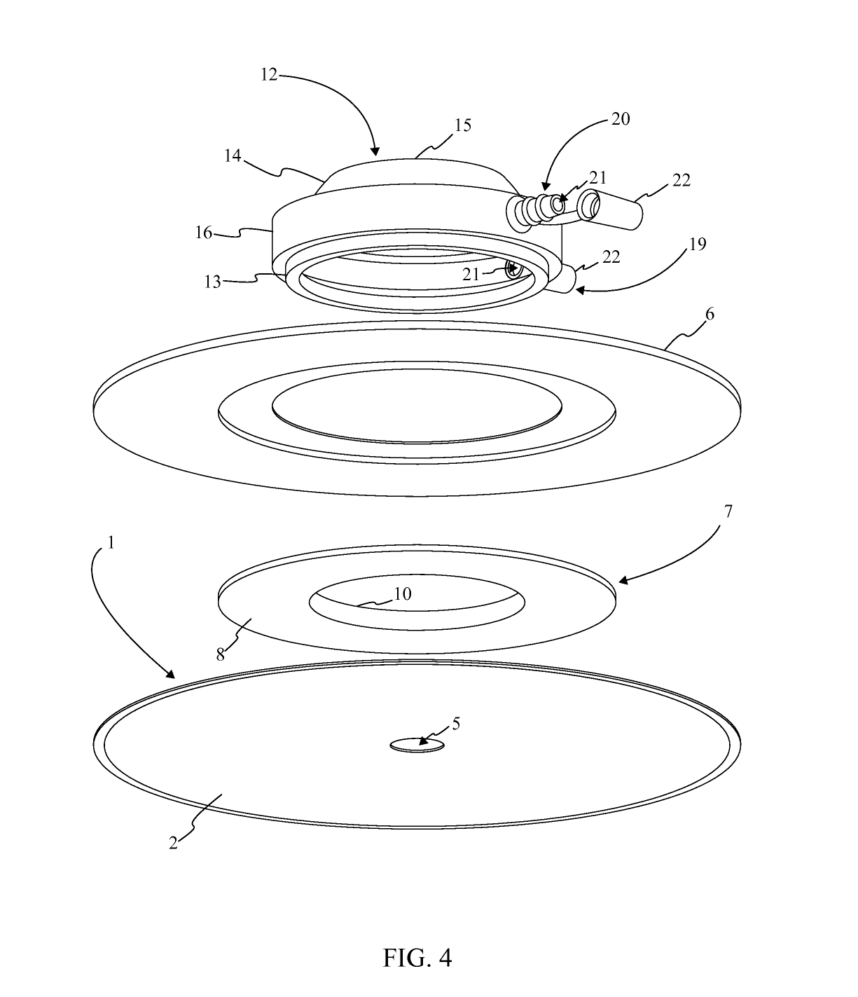

Referencing FIG. 4, the adhesive interface 2 securely fastens the wafer 1 to the user's body. In the current embodiment of the present invention, the adhesive interface 2 is a medical grade water resistant adhesive that seals the wafer 1 against the peristomal area of the user's body. The medical grade water resistant adhesive compound allows a user to keep the wafer 1 attached to their body during a shower, reducing the replacement frequency of the wafer 1.

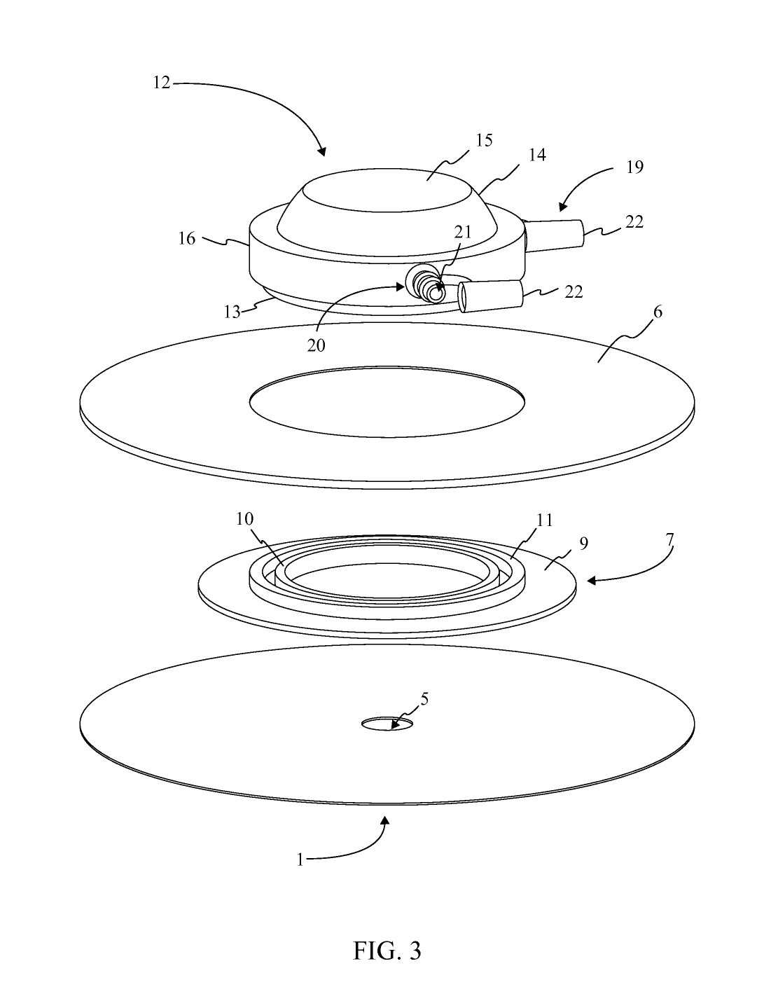

Referencing FIG. 3 and FIG. 5, the appliance mount 7 is a rigid circular component that allows the convex drainage appliance 12 to securely attach to the wafer 1. The appliance mount 7 is positioned coincident with the wafer 1 opposite the adhesive interface 2. The appliance mount 7 is positioned between the inner section 3 and the outer section 4. The appliance mount 7 is positioned concentrically to the stoma opening 5. The appliance mount 7 is sealed to the wafer 1 by way of the appliance mount coupler 6. In the current embodiment of the present invention the appliance mount 7 comprises a base section 8, a first ring mount 10, a second ring mount 11, and a flange 9. The base section 8 is a surface of the appliance mount 7 that is coincident with the wafer 1 between the inner section 3 and the outer section 4. The first ring mount 10 and the second ring mount 11 are circular protruding elements concentrically positioned to one another separate opposite the base section 8. The first ring mount 10 is found immediately adjacent to the inner section 3 while the second ring mount 11 is found immediately adjacent to the flange 9. The flange 9 is a planar section 15 of the appliance mount 7 found positioned immediately adjacent to the outer section 4 opposite the base section 8.

Referencing FIG. 1, FIG. 5, and FIG. 6, the first ring mount 10 and the second ring mount 11 function as complementarily mounting elements for securing the convex drainage appliance 12 to the appliance mount 7. The first ring mount 10 is peripherally positioned to the inner section 3 and is concentric to the stoma opening 5. The second ring mount 11 is concentric to the first ring mount 10 but spaced from the first ring mount 10 forming a gap between them. The gap between the first ring mount 10 and the second ring mount 11 function complementarily to one another in order to coincidentally engage the convex drainage appliance 12. The coincident engagement seals the convex drainage appliance 12 against the appliance mount 7 creating an enclosed space bordered by the stoma opening 5, inner section 3, the base section 8, the first ring mount 10, and the convex drainage appliance 12. It should be noted that engagement between the appliance mount 7 and the convex drainage appliance 12 can be accomplished through a plastic weld.

Referencing FIG. 3, FIG. 4, and FIG. 5, the appliance mount coupler 6 securely joins the appliance mount 7 to the wafer 1. In the current embodiment of the present invention, the appliance mount coupler 6 is an adhesive layer that engages the flange 9 and the outer section 4. The appliance mount coupler 6 fastens the flange 9 to the outer section 4, securing the appliance mount 7 to the wafer 1. The appliance mount coupler 6 spans from the edge of the second ring mount 11 to the peripheral edge of the outer section 4 of the wafer 1. The secure engagement of the appliance mount coupler 6 with the appliance mount 7 seals the base section 8 against the wafer 1 between the inner section 3 and the outer section 4. It should be noted that in additional embodiments, the base section 8 could incorporate an adhesive to form a seal with the wafer 1 in addition to the engagement provided by the appliance mount coupler 6.

Referencing FIG. 5 and FIG. 6, the convex drainage appliance 12 is a partially convex structure that engages the appliance mount 7 with the cavity of the structure disposed towards the inner section 3 and the stoma opening 5. The engagement of the convex drainage appliance 12 with the appliance mount 7 forms a sealed enclosure over the inner section 3 and the stoma opening 5. The convex drainage appliance 12 is centrally aligned with the stoma opening 5. The convex drainage appliance 12 contains features that direct excretions, such as urine, from the stoma opening 5 towards drainage ports. In the current embodiment of the present invention, the convex drainage appliance 12 comprises a mounting base 13, lateral wall 14, a planar section 15, and a fluid channel 16. The mounting base 13 is a structural element of the convex drainage appliance 12 that engages the first mounting ring and the second mounting ring forming a sealed engagement. The lateral wall 14 are curved wall sections of the convex drainage appliance 12 with the concavity of their curvature disposed towards the inner section 3 and the stoma opening 5. The curvature of the lateral wall 14 assists in directing excretions to the fluid channel 16. The planar section 15 is positioned immediately adjacent to the lateral wall 14 and found distal to the stoma opening 5 and the inner section 3. The planar section 15 provides the present invention with a concealable profile when attached to a user's body. The fluid channel 16 is a peripherally positioned duct found between the mounting base 13 and the lateral wall 14. The fluid channel 16 directs the flow of excretions, such as urine to one of the at least two drainage stems 18. The fluid channel 16 comprises an angled wall 17 that are sloped towards the center of the fluid from the mounting base 13. The angled wall 17 function complementarily with the lateral wall 14 to direct fluid towards the interior area of the fluid channel 16 where the at least two drainage stems 18 are positioned.

Referencing FIG. 5, due to the adjacent positioning of the flange 9 and subsequently the appliance mount coupler 6 to the drainage stems, the appliance mount coupler 6 is of sufficient thickness to secure the appliance mount 7 to the wafer 1 while being sufficiently thin enough in order to avoid interfering or obstructing the drainage stems. It should be noted that the adhesive utilized by the appliance mount coupler 6 is a water resistant adhesive that be similar to the medical grade water resistant adhesive utilized by the adhesive interface 2 of the wafer 1 but does necessitate the same requirements as the appliance mount coupler 6 does not engage a user's skin.

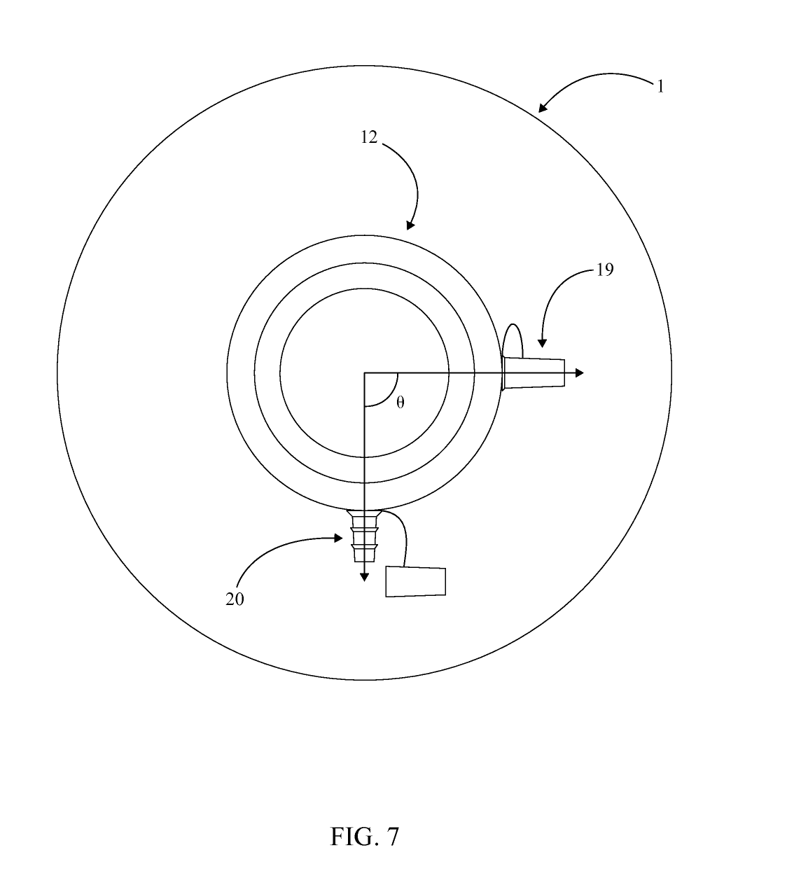

Referencing FIG. 5, FIG. 6, and FIG. 7, the at least two drainage stems 18 are fluid outlets that are in fluid communication with the fluid channel 16. The at least two drainage stems 18 are peripherally positioned on the convex drainage appliance 12, wherein the at least two drainage stems 18 traverse in the convex drainage appliance 12 by way of the fluid channel 16. The at least two drainage stems 18 allow a user to flush the spaced enclosed by the convex drainage appliance 12 reducing bacterial growth. The at least two drainage stems 18 provide a mounting point for drainage hoses that empty into a leg bag. The at least two drainage stems 18 comprise a first drainage stem 19 and a second drainage stem 20. The first drainage stem 19 and the second drainage stem 20 are particularly positioned to one another in order to allow the drainage of excretions while the user is positioned on their side. The first drainage stem 19 and the second drainage stem 20 are radially positioned about the fluid channel 16, wherein the first drainage stem 19 and the second drainage stem 20 are angularly positioned about the fluid channel 16 where the θ of their vector angle is less than 180°. In the preferred embodiment of the present invention, the first drainage stem 19 and the second drainage stem 20 are positioned at 90° angles from one another. In the preferred embodiment the first drainage stem 19 would be found in either the 3 o'clock position or the 9 o'clock positioned while the second drainage stem 20 would be found at the 6 o'clock position.

Referencing FIG. 5, FIG. 6, and FIG. 7, the first drainage stem 19 and the second drainage stem 20 each comprise a fluid conduit 21 and a tethered drainage plug 22. The first drainage stem 19 and the second drainage stem 20 are longitudinally traversed by the fluid conduit 21. The fluid conduit 21 is a drainage passage for excretions such as urine to exit the enclosed space formed by the convex drainage appliance 12. The fluid conduit 21 is in fluid communication with the fluid channel 16. Fluids collected within the enclosure created by the convex drainage appliance 12 are directed into the fluid channel 16 by the lateral wall 14 and the angled wall 17 of the fluid channel 16. Through the positioning of the user, collected excretions are directed towards into the fluid conduits 21 of the first drainage stem 19 and the second drainage stem 20. Through the attachment of a drainage hose to the first drainage stem 19 or the second drainage stem 20, the excretions flow out of the urostomy drainage appliance and into an external fluid collecting receptacle such as a leg bag. The tethered drainage plug 22 detachably seals the fluid conduit 21. The tethered drainage plug 22 is a stopper or a cap that is attached to either the first drainage stem 19 or the second drainage stem 20 through a flexible member. The tethered drainage plug 22 provides a user the ability to interchangeably connect a drainage hose between the first drainage stem 19 and the second drainage stem 20 by detachably capping the drainage stem that is not being used. Additionally the tethered drainage plug 22 maintains attached to the urostomy drainage appliance when detached from the fluid conduit 21 to allow a user to find and reattach the tether drainage plug 22 with one hand.

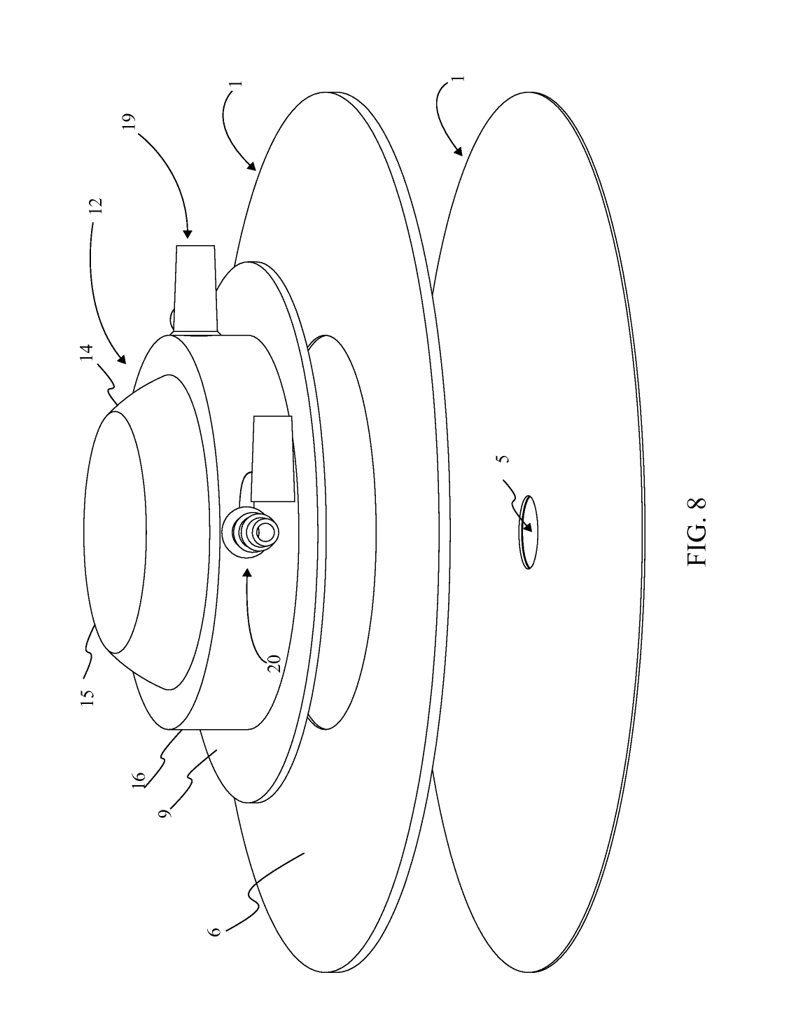

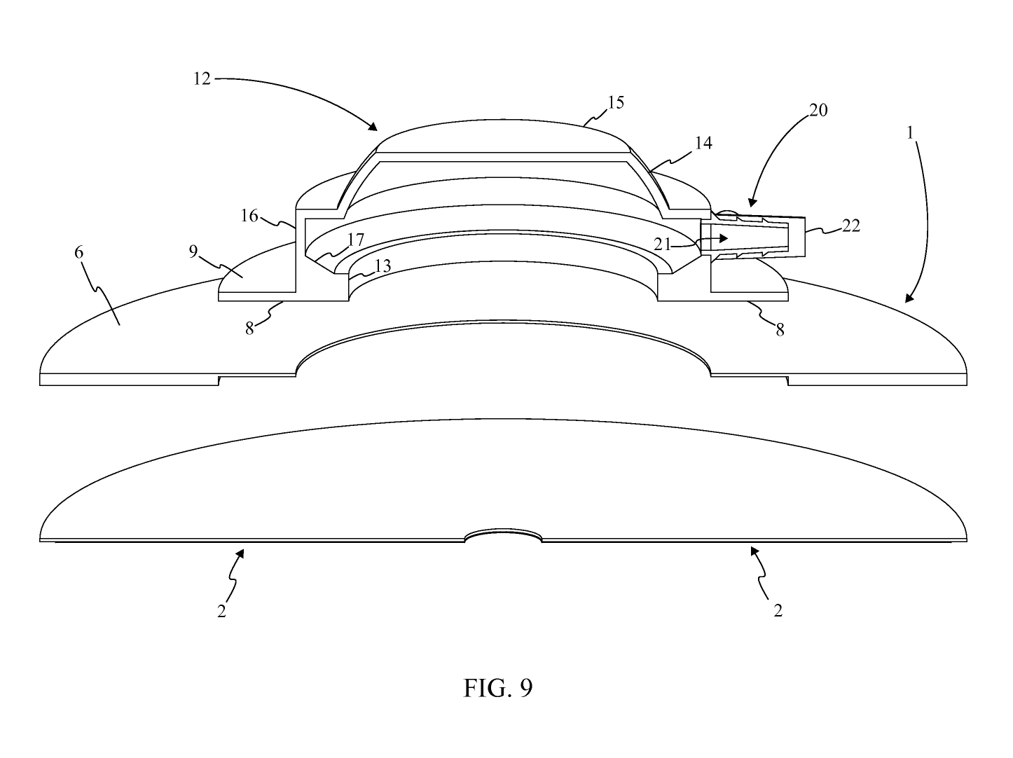

Referencing FIG. 8 and FIG. 9, in an embodiment of the present invention, the convex drainage appliance 12 is constructed with the appliance mount 7 integrally fused to the mounting base 13. In the aforementioned embodiment, the engagement of the mounting base 13 with the first ring mount 10 and the second ring mount 11 would be unnecessary as the mounting base 13 would comprise a base section 8 and a flange 9. The base section 8 and the flange 9 would retain their relationship with the appliance mount coupler 6, the inner section 3, and the outer section 4 of the wafer 1.

In an additional embodiment the urostomy drainage appliance can be configured to function with a user's stoma following surgical procedures such as colostomy, ileostomy, urostomy, and ureterostomy.

Although the invention has been explained in relation to its preferred embodiment, it is to be understood that many other possible modifications and variations can be made without departing from the spirit and scope of the invention as hereinafter claimed.

What is claimed is:

1. A urostomy drainage appliance comprises:
a wafer;
an appliance mount;
a convex drainage appliance;
at least two drainage stems;
the wafer comprises an adhesive interface, an inner section, an outer section, a stoma opening, and an appliance mount coupler;
the appliance mount comprises a base section, a flange, a first ring mount, and a second ring mount;
the convex drainage appliance comprises a mounting base, a lateral wall, a planar section, a fluid channel, and at least two drainage ports;
the drainage stems each comprise a fluid conduit and a tethered drain plug;
the fluid channel comprises an angled wall;
the appliance mount being positioned between the inner section and the outer section;
the appliance mount being concentrically positioned to the stoma opening;
the base section of the appliance mount being coincident with the wafer opposite the adhesive interface;
the appliance mount being sealed to the wafer by way of the appliance mount coupler; *
the first ring mount and the second ring mount being positioned opposite the base section;
the first ring mount being concentrically positioned between the stoma opening and the second ring mount;
the flange being circumferentially positioned to the second ring mount;
the convex drainage appliance being centrally positioned to the stoma opening;
the convex drainage appliance being sealed to the appliance mount, wherein the mounting base being coupled between the first ring mount and the second ring mount;
the fluid channel being positioned between the mounting base and the lateral wall;
the lateral wall being positioned between the fluid channel and the planar section;
the planar section being positioned distal to the stoma opening;
the lateral wall being concave to the stoma opening, wherein the concavity of the lateral wall being curved towards the stoma opening;
the fluid channel being traversed by the at least two drainage stems;
the angled wall being immediately adjacent to the mounting base;
the angled wall being sloped towards the at least two drainage stems;
the at least two drainage stems being peripherally positioned to the convex drainage appliance;
the fluid conduit traverses through each of the at least two drainage stems;
the fluid conduit being in fluid communication with the fluid channel; and
the fluid conduit being detachably sealed by the tethered drain plug.

2. The urostomy drainage appliance as claimed in claim 1 comprises:
the at least two drainage stems comprise a first drainage stem and a second drainage stem; and
the first drainage stem and the second drainage stem being radially positioned about the fluid channel, wherein the first drainage stem and the second drainage stem are angularly positioned about the fluid channel with a θ less than 180°.

3. The urostomy drainage appliance in claim 1, wherein the inner section being of a flexible material where, the flexible material is provided to accommodate stoma protrusions.

4. The urostomy drainage appliance in claim 1 wherein the adhesive interface being a medical grade water resistant adhesive.

5. The urostomy drainage appliance in claim 1, wherein the flange is secured to the outer section by way of the appliance mount coupler.

6. The urostomy drainage appliance in claim 1, wherein the appliance mount is integrally coupled to the mounting base where, the mounting base comprises a base section and a flange.

7. The urostomy drainage appliance in claim 1, wherein the base section of the appliance mount being sealed against the wafer between the inner section and the outer section.

8. A urostomy drainage appliance comprises:
a wafer;
an appliance mount;
a convex drainage appliance;
at least two drainage stems;
the wafer comprises an adhesive interface, an outer section, a stoma opening, and an appliance mount coupler;
wherein the adhesive interface being a medical grade water resistant adhesive;
wherein the inner section being of a flexible material where, the flexible material is provided to accommodate stoma protrusions;
the appliance mount comprises a base section, a flange, a first ring mount, and a second ring mount;
the convex drainage appliance comprises a mounting base, a lateral wall, a planar section, a fluid channel, and at least two drainage ports;
the drainage stems each comprise a fluid conduit and a tethered drain plug;
the fluid channel comprises an angled wall;
the appliance mount being positioned between the inner section and the outer section;
the appliance mount being concentrically positioned to the stoma opening;
the base section of the appliance mount being coincident with the wafer opposite the adhesive interface;
the appliance mount being sealed to the wafer by way of the appliance mount coupler;
the first ring mount and the second ring mount being positioned opposite the base section;
the first ring mount being concentrically positioned between the stoma opening and the second ring mount;
the flange being circumferentially positioned to the second ring mount;
the convex drainage appliance being centrally positioned to the stoma opening;
the convex drainage appliance being sealed to the appliance mount, wherein the mounting base being coupled between the first ring mount and the second ring mount;
the flange being secured to the outer section by way of the appliance mount coupler;
the fluid channel being positioned between the mounting base and the lateral wall;
the lateral wall being positioned between the fluid channel and the planar section;
the planar section being positioned distal to the stoma opening;
the lateral wall being concave to the stoma opening, wherein the concavity of the lateral wall being curved towards the stoma opening;
the fluid channel being traversed by the at least two drainage stems;
the angled wall being immediately adjacent to the mounting base;
the angled wall being sloped towards the at least two drainage stems;
the at least two drainage stems being peripherally positioned to the convex drainage appliance;
the fluid conduit traverses through each of the at least two drainage stems;
the fluid conduit being in fluid communication with the fluid channel; and
the fluid conduit being detachably sealed by the tethered drain plug.

9. The urostomy drainage appliance as claimed in claim 8 comprises:
the at least two drainage stems comprise a first drainage stem and a second drainage stem; and
the first drainage stem and the second drainage stem being radially positioned about the fluid channel, wherein the first drainage stem and the second drainage stem are angularly positioned about the fluid channel with a θ less than 180°.

10. The urostomy drainage appliance in claim 8, wherein the appliance mount is integrally coupled to the mounting base where, the mounting base comprises a base section and a flange.

11. A urostomy drainage appliance comprises:
a wafer;
an appliance mount;
a convex drainage appliance;
at least two drainage stems;
the wafer comprises an adhesive interface, an outer section, a stoma opening, and an appliance mount coupler;
wherein the adhesive interface being a medical grade water resistant adhesive;
wherein the inner section being of a flexible material where, the flexible material is provided to accommodate stoma protrusions;
the appliance mount comprises a base section, a flange, a first ring mount, and a second ring mount;
the convex drainage appliance comprises a mounting base, a lateral wall, a planar section, a fluid channel, and at least two drainage ports;
the drainage stems each comprise a fluid conduit and a tethered drain plug;
the fluid channel comprises an angled wall;
the at least two drainage stems comprise a first drainage stem and a second drainage stem;
the appliance mount being positioned between the inner section and the outer section;
the appliance mount being concentrically positioned to the stoma opening;
the base section of the appliance mount being coincident with the wafer opposite the adhesive interface;
the appliance mount being sealed to the wafer by way of the appliance mount coupler;
the first ring mount and the second ring mount being positioned opposite the base section;
the first ring mount being concentrically positioned between the stoma opening and the second ring mount;
the flange being circumferentially positioned to the second ring mount;
the convex drainage appliance being centrally positioned to the stoma opening;
the convex drainage appliance being sealed to the appliance mount, wherein the mounting base being coupled between the first ring mount and the second ring mount;
the flange being secured to the outer section by way of the appliance mount coupler;
the fluid channel being positioned between the mounting base and the lateral wall;
the lateral wall being positioned between the fluid channel and the planar section;
the planar section being positioned distal to the stoma opening;
the lateral wall being concave to the stoma opening, wherein the concavity of the lateral wall being curved towards the stoma opening;
the fluid channel being traversed by the at least two drainage stems;
the angled wall being immediately adjacent to the mounting base;
the angled wall being sloped towards the at least two drainage stems;
the at least two drainage stems being peripherally positioned to the convex drainage appliance;
the fluid conduit traverses through each of the at least two drainage stems;
the fluid conduit being in fluid communication with the fluid channel;
the fluid conduit being detachably sealed by the tethered drain plug; and
the first drainage stem and the second drainage stem being radially positioned about the fluid channel, wherein the first drainage stem and the second drainage stem are angularly positioned about the fluid channel with a θ less than 180°.

12. The urostomy drainage appliance in claim 11, wherein the appliance mount is integrally coupled to the mounting base where, the mounting base comprises a base section and a flange.
